(19) United States
(12) Patent Application Publication
(10) Pub. No.: US 2020/0060434 A1
(43) Pub. Date: Feb. 27, 2020

(54) CHILD-SUPPORTING HARNESS

(71) Applicant: BEBE URBAIN INC., Saint-Majorique-de-Grantham (CA)

(72) Inventors: Marie-Eve CARON, Saint-Majorique-de-Grantham (CA); Martin LABERGE, Montreal (CA); Bastien JOURDE, Montreal (CA); Philippe LEFEBVRE, Montreal (CA); Patrick BOIRE-SCHWAB, Verdun (CA); Christine CHARLEBOIS, Montreal (CA); Stéphane BEAULÉ, Saint-Majorique (CA); Kristina PROULX, Drummondville (CA)

(21) Appl. No.: 16/488,014

(22) PCT Filed: Feb. 23, 2018
(86) PCT No.: PCT/CA2018/050210

§ 371 Date: Aug. 22, 2019

Related U.S. Application Data
(60) Provisional application No. 62/462,384, filed on Feb. 23, 2017.

(51) Int. Cl. A47D 13/02; A61B 5/0205; A61B 5/00

(52) U.S. Cl. CPC A47D 13/025; A61B 5/024; A61B 5/746; A61B 5/02055

(57) ABSTRACT

A child-supporting harness comprises a harness portion and a child-carrying portion. The harness portion comprises a dorsal member having an upper section and a lower section; a waist strap depending from the lower section of the dorsal member; a pair of shoulder straps depending from the upper section of the dorsal member; and a pair of torso straps selectively attached to the dorsal member between the upper section and the lower section thereof. The child-carrying pouch is releasably secured to the harness portion.

### CROSS-REFERENCE TO RELATED APPLICATION

This application claims priority from U.S. patent provisional application 62/462,384 filed Feb. 23, 2017, the specification of which is hereby incorporated herein by reference in its entirety.

### BACKGROUND

### (a) Field

The invention relates to a harness and more specifically to child-supporting harness forming a child-carrying pocket or pouch for carrying a child.

### (b) Related Prior Art

Child-supporting harnesses have long been known in the art and are available in many different configurations. Existing child-supporting harnesses are constructed to support the child on the chest-side of the user and the harness normally comprises a bag-like support and two or more looped harness straps, one for each shoulder on the rear side of the bag for the wearing of the child-supporting harness on the shoulders.

This kind of child-supporting harness is encumbered with limitations which the present child-supporting harness aims to overcome.

### SUMMARY

According to an embodiment, there is disclosed a child-supporting harness comprising: a harness portion comprising: a dorsal member having an upper section and a lower section; a waist strap depending from the lower section of the dorsal member; a pair of shoulder straps depending from the upper section of the dorsal member; a pair of torso straps selectively attached to the dorsal member between the upper section and the lower section thereof; and a child-carrying pouch releasably secured to the harness portion.

According to an aspect, the pair of torso straps is selectively attached to the dorsal member at a position which is adjustable between the lower section and the upper section of the dorsal member.

According to an aspect, each torso strap of the pair of torso straps crosses a corresponding shoulder strap of the pair of shoulder straps.

According to an aspect, one of the shoulder straps and the torso straps comprise a slot, with the other of the shoulder straps and the torso straps passes through the slot when crossing each other.

According to an aspect, each torso strap of the pair of torso straps comprises a free end which is unattached to other part of the harness portion.

According to an aspect, the free end of each torso strap of the pair of torso straps comprises a base fixation used to releasable secure the child-carrying pouch.

According to an aspect, the child-carrying pouch comprises a seat support section and separate head support section.

According to an aspect, the seat support section is pre-molded to substantially match a shape of a child's buttocks area.

According to an aspect, the separate head support section is adjustable at least in height relative to the seat support section.

According to an aspect, the child-supporting harness further comprises sensors for sensing at least one of temperature, cardiac frequency and respiratory rate of a child in the child-carrying pouch.

According to an aspect, the sensors are further adapted to sense one or more of the child's weight, the child's oxygenation level, the time spent in the carrier by the child, as information on the position of the child in the child carrying pouch (e.g. using pressure sensors) and movements of child during the time spent in the child carrying pouch.

According to an aspect, the child-supporting harness comprises a strip comprising the sensors for contacting the skin of the child when in the child-carrying pouch.

According to an aspect, the head supporting section comprises a main area and a strip, the strip extending from the main area and comprising the sensors, wherein the strip is for contacting the skin of the child when in the child-carrying pouch.

According to an aspect, the child-supporting harness further comprises a signalization device located in the harness portion and in communication with the sensors, the signalization device generating a signal when at least one of the temperature, the cardiac frequency and the respiratory rate meet an alarm criterion.

According to an aspect, the signalization device comprises one of a vibration device generating a vibration signal, a lighting device generating a light signal, and a speaker generating a sound signal

According to an aspect, the vibration device is located in at least one of the shoulder straps.

According to an aspect, the dorsal member comprises one of rigid material and semi-rigid material.

According to an aspect, the dorsal member comprises a first section and a second section, and a length, and wherein the first section is adjustable relative to the second section to adjust the length of the dorsal member.

According to an aspect, the harness portion comprises a front and a back which respectively correspond a front and a back of a user wherein the child-supporting harness further comprises additional the base fixations located on the front and on the back of the harness portion, thereby allowing the child-carrying pouch to be secured both to the back and to the front of the harness portion.

According to an aspect, the child-carrying pouch comprises another child-carrying pouch releasably secured to the harness portion, thereby resulting in having one child-carrying pouch releasably secured to front of the harness portion and the other child-carrying pouch releasably secured to back of the harness portion.

According to an aspect, the child-carrying pouch comprises a seat supporting section and a sitting-width about the seat-supporting section, wherein the child-carrying pouch comprises an adjusting means to adjust the sitting-width of the child-carrying pouch.

According to an aspect, the child-carrying pouch comprises a non-stretching area and a stretching area surrounded by the non-stretching area.

According to an embodiment, there is described a child-supporting harness comprising a child-carrying pouch comprising a layer of material having sensors for sensing a biometric parameter of a child in the child-carrying pouch.

According to an aspect, the biometric parameter comprises at least one of temperature, cardiac frequency, weight and respiratory rate.

According to an aspect, the layer of material of the child-carrying comprises a main area and a strip, the strip extending from the main area and comprising the sensors, wherein the strip is for contacting the skin of the child of the child when in the child-carrying pouch.

According to an aspect, the child-supporting harness further comprises a vibration device connected to the sensors, the vibration device vibrating when at least one of the temperature, the cardiac frequency and the respiratory rate meet an alarm criterion.

According to an aspect, the child-supporting harness further comprises shoulder straps wherein the vibration device is located in at least one of the shoulder straps.

According to an embodiment, there is disclosed a child-supporting harness comprising a child-carrying pouch comprising a seat support section which is pre-molded to substantially match a shape of a child's buttocks area.

According to an aspect, the child-carrying pouch comprises a head support section separate from the seat support section.

According to an aspect, the separate head support section is adjustable at least in height relative to the seat support section.

### BRIEF DESCRIPTION OF THE DRAWINGS

Further features and advantages of the present disclosure will become apparent from the following detailed description, taken in combination with the appended drawings, in which:

FIG. 1 is a schematic of a child-supporting harness worn by a user for carrying a child in accordance with an embodiment;

FIG. 2 is a perspective view of the back of the child-supporting harness as worn by a user, with only the straps visible;

FIG. 3 is a schematic of a seat support section for installation in a child-supporting harness in accordance with an embodiment;

FIG. 4 is a perspective view of the back of the child-supporting harness as worn by a user, with the user invisible;

FIG. 5 is a perspective view of the back of the child-supporting harness as worn by a user, with the user and the near panel invisible;

FIG. 6 is a view of some unassembled pieces to be assembled to form the child-supporting harness, and a sub-assembly of pieces forming the straps in accordance with an embodiment;

FIGS. 7 and 8 are image showing images of various embodiments of child-supporting harnesses worn by a user;

FIG. 9 is an elevated perspective view of a child-supporting harness worn by a user to carry a child in front fashion according to an embodiment;

FIG. 10 is a front perspective view of the child-supporting harness shown on FIG. 9 as worn by another user;

FIG. 11 is a left-side perspective view of the child-supporting harness shown on FIGS. 9 and 10 with two children held, one in a front fashion and one in a back fashion;

FIG. 12 is a front perspective view of the harness of the child-supporting harness shown on FIGS. 9 to 11 as worn by a user without a child-carrying pouch;

FIG. 13 is a rear perspective view of the harness of FIG. 12;

FIG. 14 is a right-side perspective view of the harness of FIGS. 12 and 13;

FIG. 15 is a rear view of the rear portion of the harness shown on FIGS. 12-14 alone;

FIG. 16 is a front view of the front portion of the harness shown on FIGS. 12-14 alone;

FIG. 17 is a rear view of the front portion of the harness shown on FIG. 16;

FIG. 18 is a front perspective view of the child-supporting harness of FIGS. 9-11 as worn by a user with a child-carry pouch worn in a front fashion;

FIG. 19 a left-side perspective view of the child-supporting harness of FIGS. 9-11 as worn by a user with a child-carrying pouch worn in a front fashion and a child-carrying pouch worn in a back fashion;

FIG. 20 is a rear view of the harness of FIGS. 12-14;

FIG. 21 is a right-side perspective view of the child-supporting harness of FIGS. 9-11 as worn by a user with a child-carrying pouch worn in a back fashion;

FIG. 22 is an exploded perspective view of the child-carrying pouch according to an embodiment;

FIG. 23 is an assembled view of the child-carrying pouch of FIG. 22;

FIG. 24 is a schematic view of a combination of the child-support harness of an embodiment and a monitoring device.

FIG. 25 is a front perspective view of a child-supporting harness according to another embodiment worn by another user;

FIG. 26 is a right-side perspective view of the child-supporting harness of FIG. 25 worn by a user with a child-carrying pouch worn in a front fashion;

FIG. 27 is a front perspective view of a child-supporting harness according of FIGS. 26-26 with illustration of the course of the shawl forming the pouch;

FIG. 28 is a right-side perspective view of the harness portion of the child-supporting harness of FIGS. 25-27 with illustration of the course of the shawl forming the pouch; and

FIG. 29 is a closer front perspective view of the child-supporting harness of FIGS. 25-28 and an exemplary fitting used to pass portion of the shawl through.

It will be noted that throughout the appended drawings, like features are identified by like reference numerals.

### DETAILED DESCRIPTION

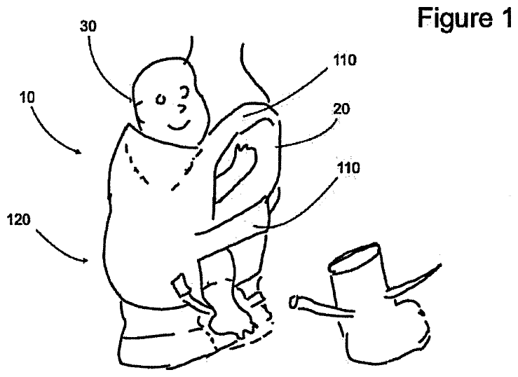

Referring now to the drawings, and more particularly to FIG. 1, the child-supporting harness 10 is worn by a user 20 to carry a child 30 in a pouch-like space or compartment wherein the child 30 is secure. The child-supporting harness 10, as illustrated, comprises openings on both sides through which the arms and the legs of the child 30 may extend out of the child-supporting harness 10.

The child-supporting harness 10 comprises two shoulder straps 110 intended to extend from the child-carrying pouch 120 of the child-supporting harness 10 over the shoulders of the user 20 down the back of the user 20 and back to the front of the user 20 at the bottom of the child-carrying pouch 120 at the front of the user 20.

The shoulder straps 110, based on embodiments, are either crossing at the back of the user 20, or not. The shoulder straps, according to an embodiment, are permanently secured to the front portion of the child-supporting harness 10. According to an embodiment, the shoulder straps 110 are temporarily secured to the front of the child-supporting harness 10. The shoulder straps may be temporarily secured to the front of the child-supporting harness 10 at the bottom, at the waist level, or at a higher level, for example at the location wherein the shoulder straps 110 join to the front portion of the child-supporting harness 10. The shoulder straps 110 may be secured to the main portion of the child-supporting harness 10 using releasable means such as openable auto-locking devices, a belt buckle or the like, a system of hooks and eyes, rivets, buttons, releasable means such as Velcro®, and any combination thereof. The releasable means may for example be embodied as a combination of two complementary buckle components with at least one of the buckle components comprising biased parts to automatically lock together the buckle components when inserted one with the other and to release the lock when someone is pressing against a portion of at least one of the complementary buckle components to release them from each other.

According to embodiments, the shoulder straps 110 further comprises adjusting means to adjust the length of the shoulder straps 110 to maximize comfort of the user 20 and/or security of the child 30. The adjustment means may further comprise means to move closer the shoulder straps 110 at some point.

One must note that even if the child 30 illustrated on FIG. 1 is facing towards the user 20, the child-supporting harness 10 is adapted to carry a child 30 either facing toward the user 20 or facing in front in the same direction as the user 20. The selection of the orientation of the child 30 depends on multiple factors, including the preferences of the user 20, the preferences of the child 30, and the age of the child 30 which set some limitation based in part on the capacity of the child 30 to sit upright.

It is also envisaged that the child-supporting harness 10 can be worn on the back of the user 20 instead of the front of the user 20.

Still referring to FIG. 1, it is shown that the child-supporting harness 10 allows the child 30 to wear additional clothes components (boots, mittens, etc.), with the child-supporting harness 10 comprising securing means adapted to temporarily secure the clothes components to the front portion of the child-supporting harness 10. Similarly, the above-described securing means may be used to temporarily secure the clothes components to the child-supporting harness 10, such as a securing strap having on at least one of its end one securing means and the other ends being temporarily or permanently secured to the front portion of the child-supporting harness 10.

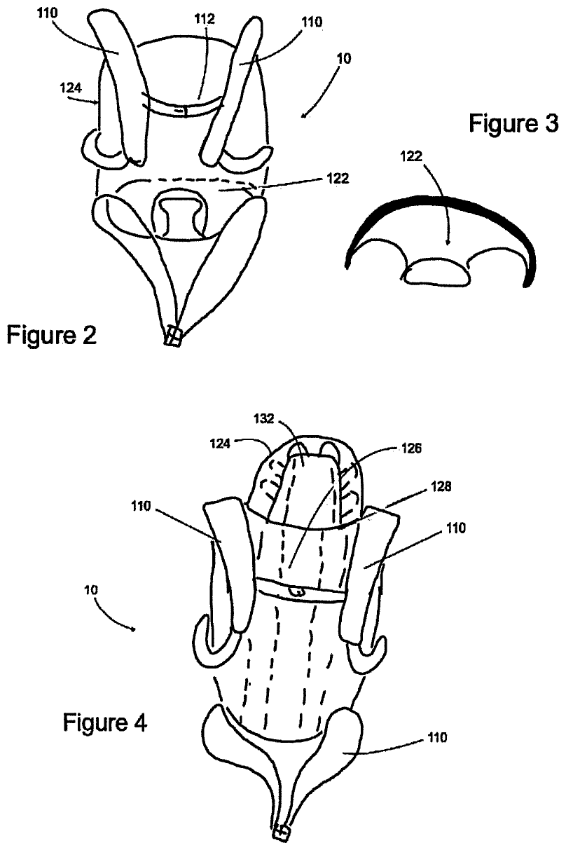

FIG. 2 shows a view of the child-supporting harness 10 from an observer located back to the user 20, with the user 20 being transparent and only some parts of the child-supporting harness 10 visible. According to the illustrated embodiment, the shoulder straps 110 keep the back of the user 20 free, and are secured close to each other through an adjustable releasable tightening strap 112 allowing both to prevent the shoulder straps 110 from slipping away from each other and therefore from the shoulders of the user 20 and to adjust the distance between the shoulder straps 110.

The child-supporting harness 10 further shows a removable seat support section 122 disposed on the floor of the child-carrying pouch 120 of the child-supporting harness 10. The seat support section 122 helps the child 30 in sitting correctly in the child-carrying pouch 120 of the child-supporting harness 10, and prevents the child 30 from slipping on one side or the other.

Additionally, referring to FIG. 3, the seat support section 122 has a curved shape with upwardly curved edges at locations corresponding to the back and between the legs of the child 30. This shape helps the child 30 to sit straight and to keep a good position in general. The seat support section 122 is removable and replaceable as the child 30 grows, to adjust to the different phases of the child development.

Now referring to FIG. 4, the illustration shows the child-supporting harness 10 from the same perspective as FIG. 2. The child-supporting harness 10 comprises to form its child-carrying pouch 120 a near panel 124, close to the user 20, and a far panel 126, away from the user 20. The near panel 124 and the far panel 126 are secured together at the bottom by a zipper or a similar attaching means able to support the weight of the child 30. The near panel 124 and the far panel 126 are secured together on the sides with securing means (such as Velcro® or rivets) providing the openings for the legs and arms of the child 30.

In addition to the shoulder straps 110 used to hold the child-supporting harness 10 close to the body of the user 20, the child-supporting harness 10 further comprising waist straps 114. The waist straps 114 are adjustable and attachable.

The near panel 124 comprises attaching means 128 at the top for securing a replaceable bib panel (not shown). The bib panel can be removed to be cleaned, and/or replaced with a new one.

The far panel 126 is adapted for receiving a head support 132 designed to help a child 30 to have its head straight. The head support 132 comprises semi-rigid ribs covered with padded fabric to support, protect and be comfortable to the child 30.

According to another embodiment, the child-supporting harness 10 comprises e-textile (a.k.a. smart textile, smart fabric, smart clothing or electronic textile) in contact with the child 30 and/or the child-carrying pouch 120.

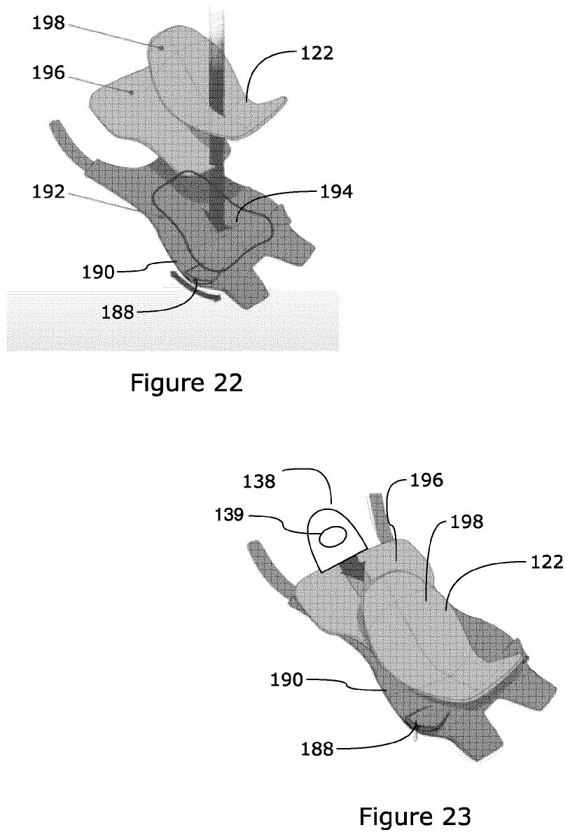

According to an embodiment, either one or both the near panel 124 and the far panel 126 comprises e-textile (aka electronic textile). E-textile as used herein is meant to include any fabric in which digital components, electrical components or electronics are embedded. According to an embodiment, the e-textile consists in fabric and/or garment components comprising electronic components acting as sensors or detectors and which are able to communicate with a memory 228, processing component 226 and/or monitor (see FIG. 22) to transmit data collected to a monitoring device 222.

According to embodiments, the e-textile can monitor a biometric parameter. In an embodiment, the biometric parameter comprises at least one of temperature of the child 30, the cardiac frequency of the child 30, the respiratory rate of the child 30, the humidity level of the child-carrying pouch 120, sound-based data, etc.

According to an embodiment, the monitoring device 222 can be secured, temporarily or not, in one of the shoulder straps 110 with the e-textile and the monitoring device 222 connected together through wiring.

According to an embodiment, the e-textile is in communication with a generic monitoring device 222 owned by the user (e.g. a smart phone) through a wireless communication technology such as Bluetooth® or through Near Field Communication (NFC) technology.

According to an embodiment, the generic monitoring device 222 requires the installation of specific software (e.g. a specialized app for a iPhone™) to enter in communication with the e-textile and to collect data from the e-textile. According to an embodiment, the specific software is programmed to detect specific conditions (e.g. the respiratory rate of the child 30 decreasing under a specific rate) and to generate alerts when one such specific condition is detected.

According to an embodiment, components of the e-textile are removable from the housing component of the child-supporting harness 10.

According to an embodiment, data collected by the monitoring device 222 (see FIG. 22) of the child-supporting harness 10 transmits the collected data to a secured website over Internet and used to participate in a m-Health process. A health evaluation program is associated with the website and collects and transmits data from and to multiples sources to generate a health profile of the child 30. According to an embodiment, the health profile allows to set custom alerts on the specialized program, for example when a heart malformation condition is associated with a child 30. Similarly, a physician may access data and may be signaled when alerts occur for such a child 30.

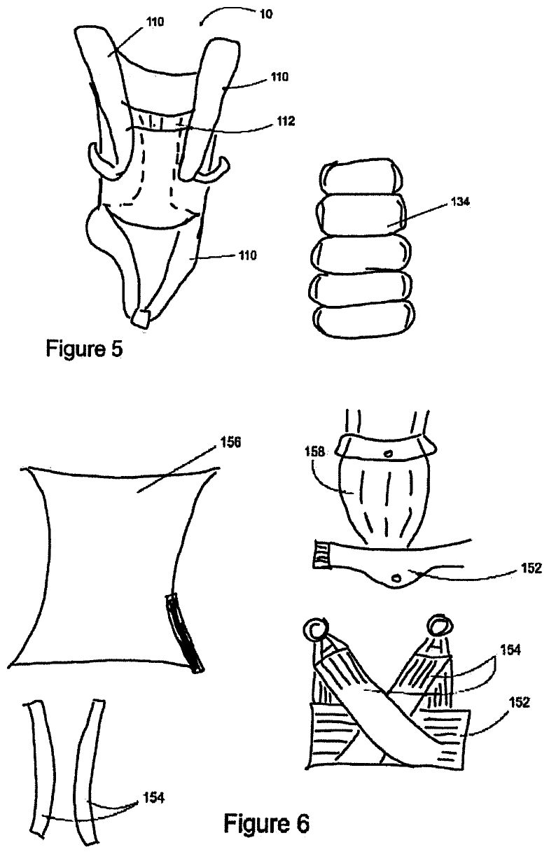

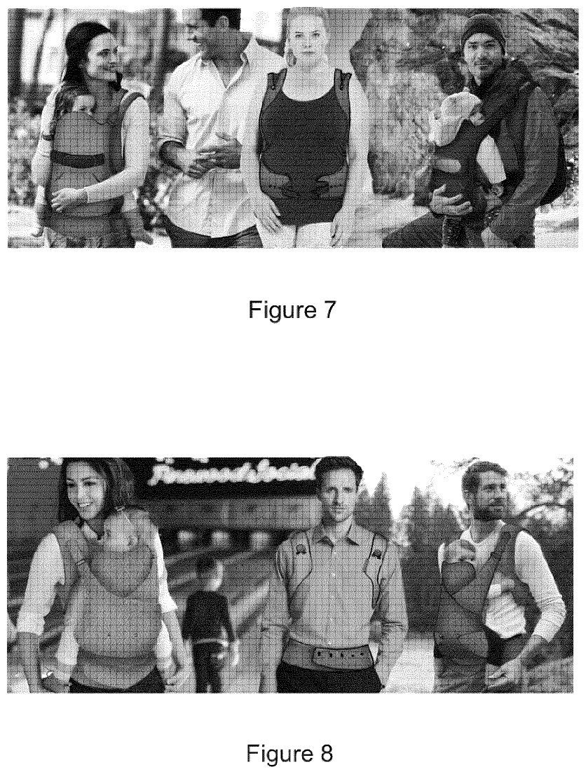

Now additionally referring to FIG. 5, the child-supporting harness 10 comprises on the side of the child-carrying pouch 120, on both sides of the far panel 126, a cushion compartment comprising a series of inflatable cushions 134 that are more or less filled with air, based on need, to provide side support to the child 30. The cushion compartments are located to not obstruct the openings for the arms and legs of the child 30. The inflatable cushions 134 of the cushion compartment consist, according to an embodiment, in a series of substantially horizontally set cushions secured together in a single cushion assembly; one cushion assembly being installed on each side of the child-supporting harness 10. According to an embodiment, the cushion assemblies are removable from the cushion compartments. According to an embodiment, the cushions of a cushion set are connected to each other, so that they are blown up together.

Referring to FIG. 6, the child-supporting harness 10 comprises a belt part 152, two shoulder strap parts 154 (forming the shoulder straps 110), a base panel part 156 (forming the near panel 124, and a carrying panel part 158 (forming the far panel 126). Some of the parts 152, 154, 156, 158 comprises a series of layers (ex. multi-layer shoulder straps, layers to form the cushion compartment, etc.). Some parts 152, 154, 156, 158 also comprise additional components (e.g., a zipper portion on the belt part, attaching means, adjusting means, ribs, e-textile, compartments, etc.).

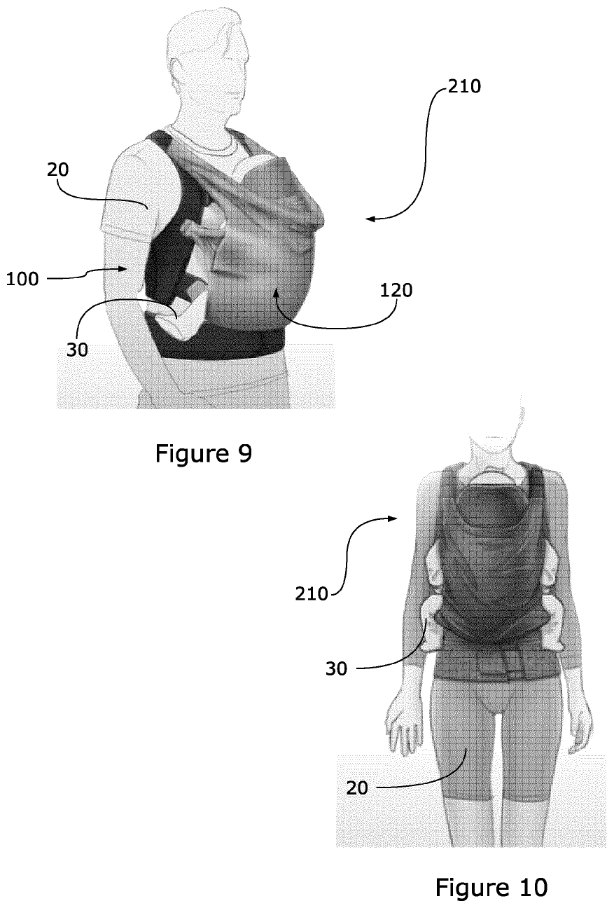

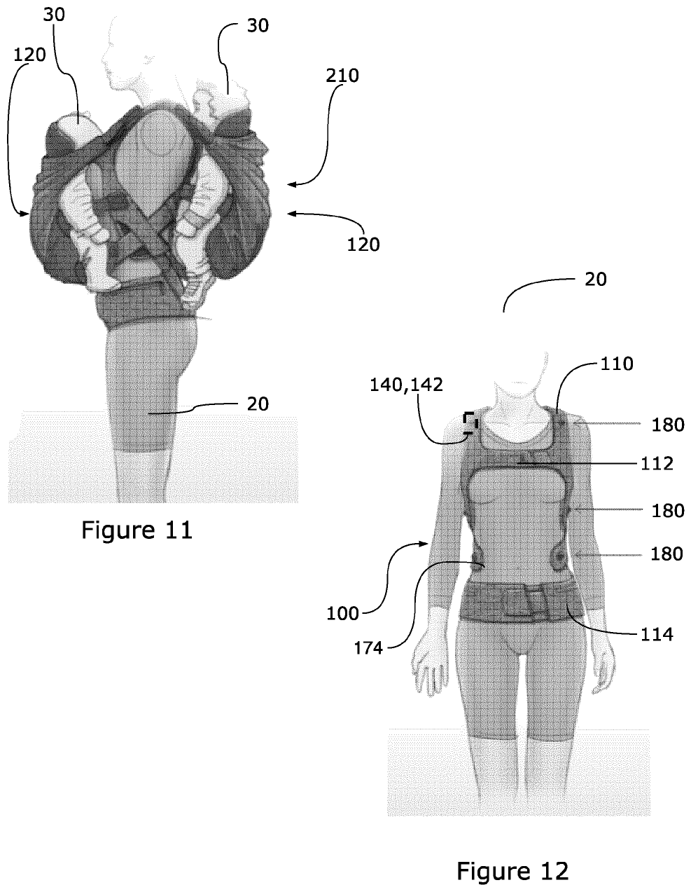

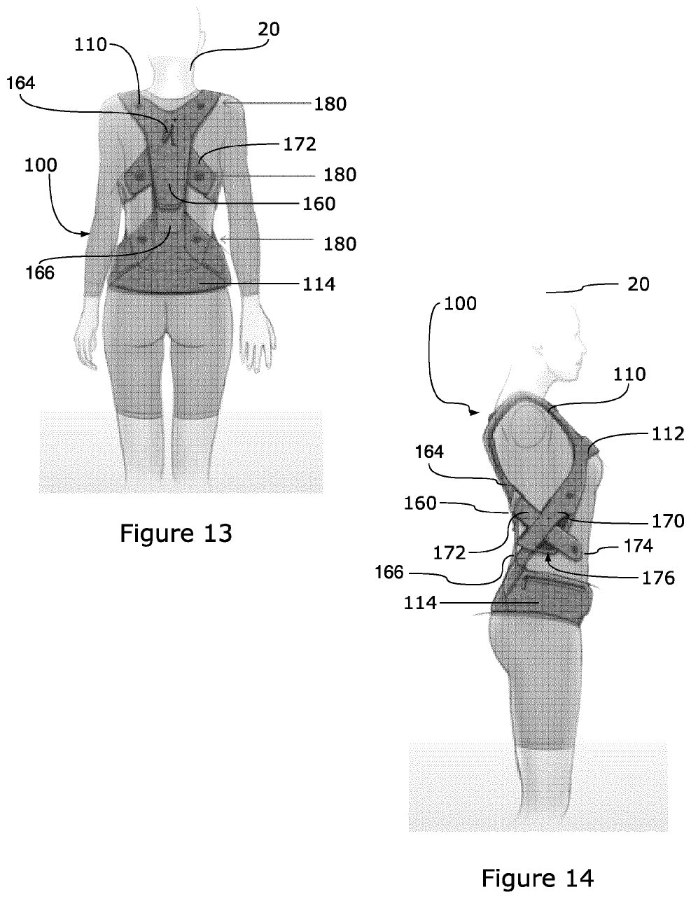

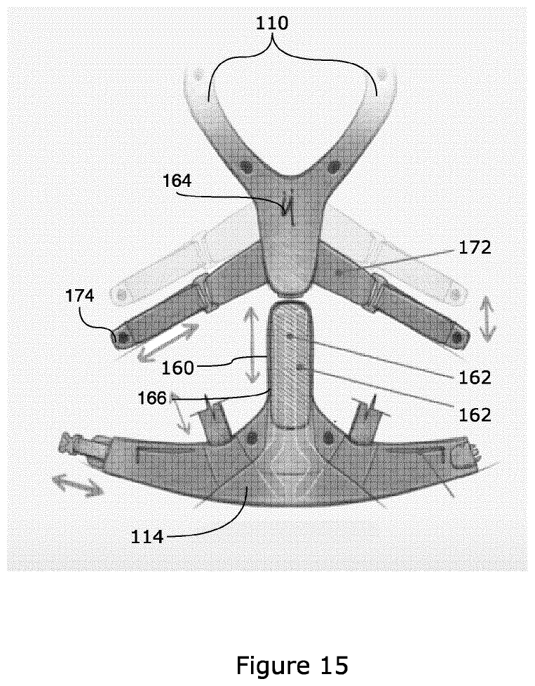

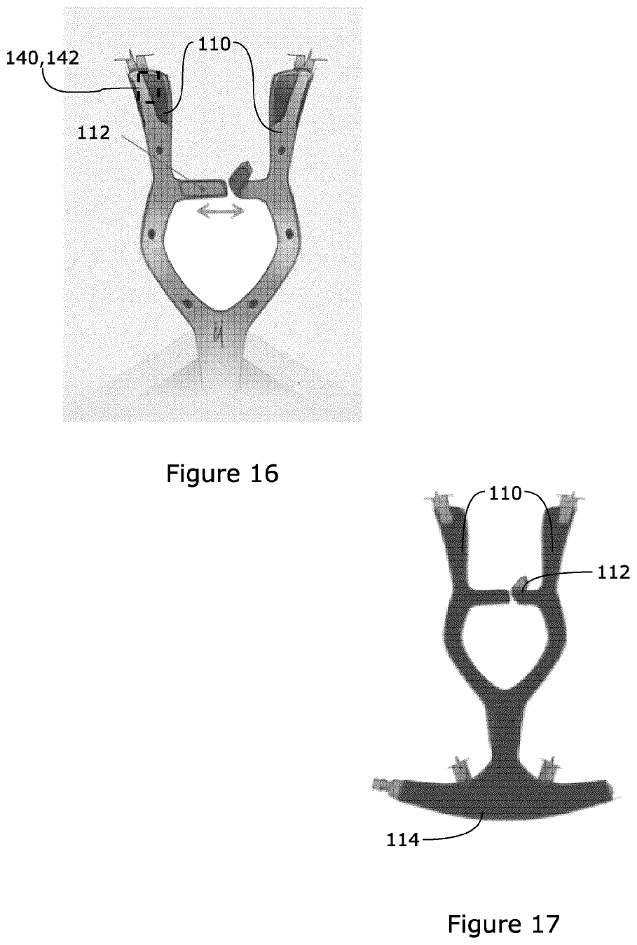

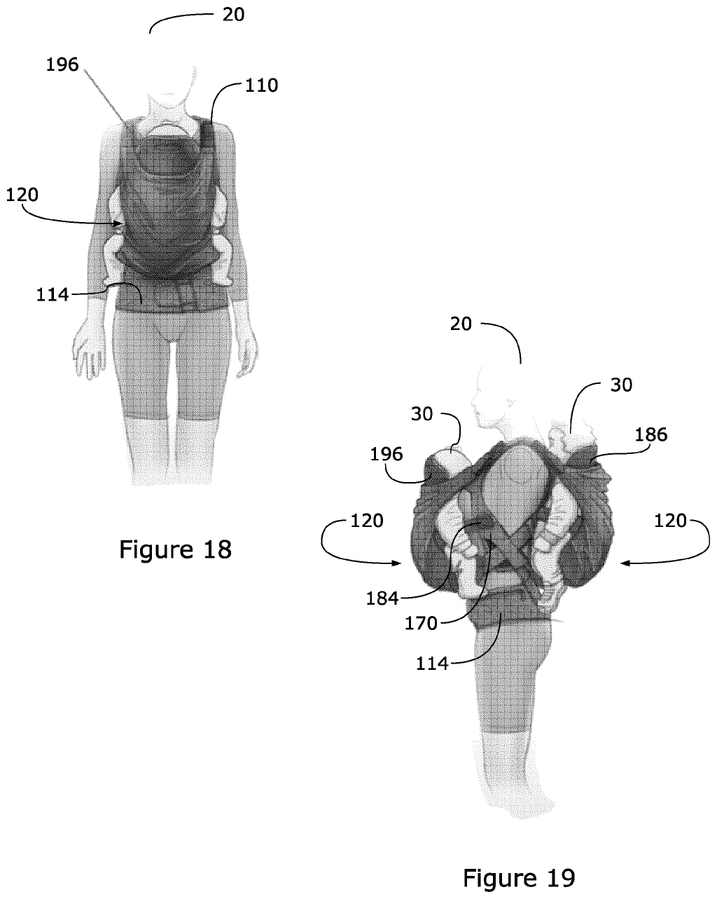

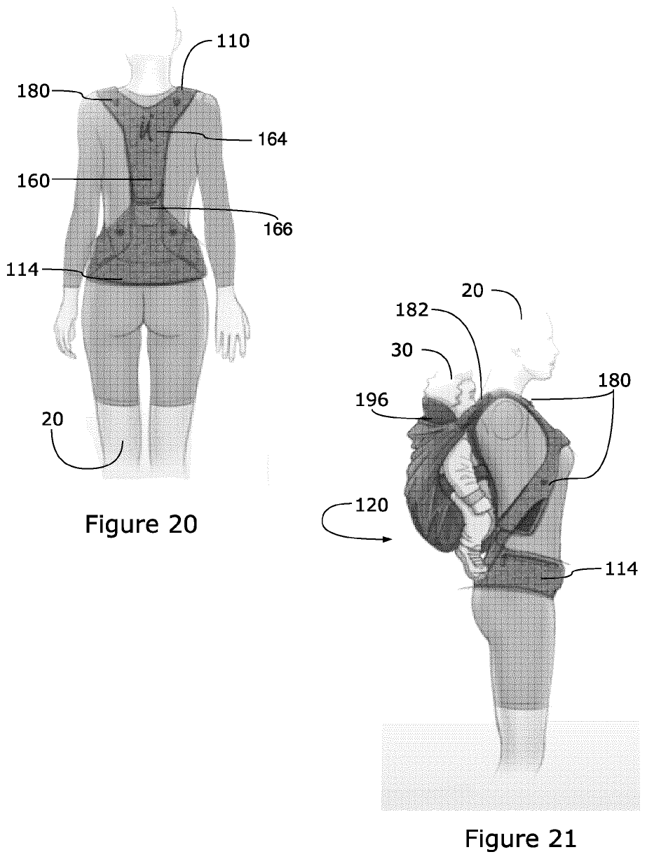

Referring now to FIGS. 9 to 21, a child-supporting harness 210 comprises a harness portion 100 comprising shoulder straps 110 to be worn over the shoulders of the user 20. The shoulder straps 110 are to be joined at the front of the user 20 (see FIGS. 12, 16 and 17) using the tightening strap 112. The tightening strap 112 comprises a securing means, for instance Velcro™′ at the end of releasable portions of the tightening strap 112 to attach one end to the other in an adjustable fashion.

The child-supporting harness 210 comprises a dorsal member 160 extending downward from the shoulder straps 110 on the back of the user 20, thus the shoulder straps 110 depending from the upper section 164 of the dorsal member 160. The dorsal member 160 has an upper section 164 and a lower section 166. The dorsal member 160 comprises an integrated structure 162 made of rigid or semi-rigid material to provide structure to the child-supporting harness 210 as to be able to transfer efforts from one end of the dorsal member 160 to the other end of the dorsal member 160. One end of the dorsal member 160 joins to the shoulder straps 110 where they join, defining a Y-shaped junction point where load is transferred in a balanced manner between the shoulder straps 110 and the dorsal member 160.

The child-supporting harness 210 comprises pectoral sections 170 of the shoulder straps 110 extending downward and sideward from the upper section of the shoulder straps 110 at the front toward the side of the user 20. According to an embodiment, the pectoral sections 170 are continuous from the main section of shoulder straps 110 and thereby are considered as sections of the shoulder straps 110.

The child-supporting harness 210 comprises torso straps 172 selectively attached to the dorsal member 160 between the upper section 164 and the lower section 166 thereof and adjustable in height relative to the dorsal member 160 between the lower section 166 and the upper section 164. The torso straps 172 are separate and distinct from the dorsal member 160, the shoulder straps 110 and the waist strap 114, and is selectively attached to the dorsal member 160 at a selected position. The torso straps 172 extend downward and sideward from the shoulder straps 110 at the back toward the side of the user 20. The pectoral sections 170 and the torso straps 172 cross each other about the side of the user 20. More precisely, each torso strap 172 of the pair of torso straps 172 crosses a corresponding pectoral strap 170 of the pair of pectoral sections 170. The pectoral sections 170 comprises a slot 176 defining a passage for the torso straps 172 to pass through, providing adjustable support through the combination of the pectoral sections 170 and the torso straps 172. According to an embodiment not shown, the torso straps 172 extend to join the waist strap 114. According to embodiments, the slot 176 provides an aperture wherein the free end 174 of the torso straps 172 may be placed temporarily when not is used, or the slots 176 is designed to blocks the pectoral sections 170 to prevent the pectoral sections 170 to slide relative to the torso straps 172 when in use.

The torso straps 172, according to an embodiment, are removable from the child-supporting harness 210, being integrated only when appropriate, namely when the child-carrying pouch 120 is worn in a front fashion. The torso straps 172 are further adjustable in height, capable of being sled vertically relatively to the dorsal member 160, to adjust to the height of the user 20. According to an embodiment, each torso strap 172 of the pair of torso straps 172 comprises a free end 174 which is unattached to other part of the harness portion 100. According to an embodiment, the torso straps 172 come as a pair. The pair of torso straps 172 form a V-shape that is inversed when installed on the dorsal member 160.

The child-supporting harness 210 comprises waist strap 114 depending from the lower section 164 of the dorsal member 160 on the back, to the pectoral sections 170 about one of the side and the back of the user 20, and to the torso straps 172 about one of the side and the back of the user 20. The waist strap 114 are to be joined in front of the user 20 to be worn by the user 20 around the waist. As with the tightening strap 112, the waist strap 114 comprises adjustable securing means to secure the waist strap 114 around the waist of the user 20 while being adjustable to variable waist sizes.

The child-supporting harness 210 further comprises base fixations 180 located on at least one of the shoulder straps 110, the dorsal member 160, the waist strap 114, the pectoral sections 170 and the torso straps 172. According to an embodiment, the base fixations 180 are located at the front and at the back of the child-supporting harness 210, allowing the user 20 to wear the child-carrying pouch 120 both in a front fashion, in a back fashion, or to have two child-carrying pouches 120 secured to the harness portion 100. According to an embodiment, the base fixations 180 are located at three distinct heights of the harness portion 100: at the base with the base fixations 180 being located on the waist strap 114, the pectoral sections 170 or the torso straps 172; about at the mid-height of the harness portion 100 with the base fixations 180 being located on the torso straps 172 or the pectoral sections 170; and at the top of the harness portion 100 with the base fixations 180 being located on the shoulder straps 110. The base fixations 180 may be embodied in different forms, for example anchoring means, tying means, Velcro™ and hook means, magnet-based fixtures and side release fixtures. The selection of the type of base fixations 180 being used is based on the desired weight the base fixations 180 must support, the easiness to secure and to unsecure them as the security of the fixation solution against accidental unmounting.

The child-supporting harness 210 further comprises a child-carrying pouch 120 for carrying a child 30. The child-carrying pouch 120 defines a child-carrying pouch to hold the child 30 into, and complementary fixations 182 to be coupled to the base fixations 180 to releasably secure the child-carrying pouch 120 to the harness portion 100.

According to an embodiment, the complementary fixations 182 comprises adjustable components 184 to adjust the position of the child-carrying pouch 120 relative to the harness portion 100, thus adjusting the position of the child 30 relative to the user 20.

According to an embodiment, the child-carrying pouch 120 has a sitting-width about where the child 30 held in the child-carrying pouch 120 would sit. The child-carrying pouch 120 comprises an adjusting means 188 to adjust the sitting-width of the child-carrying pouch 120, thus providing additional control to the user 20 over the height, the closeness and other elative position characteristics of the child 30 relative to the user 20, as the position of the child 30 in the child-carrying pouch 120.

Referring now additionally to FIGS. 22 and 23, the child-carrying pouch 120 of the child-supporting harness 210 comprises an external layer 190, a head support section 196 and an inner layer 198. The inner layer 198 consists in a removable pre-shaped molded layer comprising rigid or semi-rigid material that may be shaped specifically to fulfil the needs of the child 30 to be carried. According to an embodiment (not shown), the pre-shaped inner layer 198 is made of memory foam, a combination of memory foam and a cover, or a combination of a rigid or semi-rigid material shaped as needed, a soft material applied over the shaped material, and a cover. According to an embodiment, the cover is removable from the combination to be washed. According to an embodiment, the shape of the pre-shaped molded layer is obtained through the assembly of sub-sections of the layer which, when assembled together according to a predetermined pattern, result in some of the sub-sections being in non-planar configurations with each other.

The head support section 196 and the inner layer 198 are adjustable relative to each other and to the outer layer 190, allowing to adjust their positions to improve the comfort of both the user 20 and the child 30, as to adjust as the child 30 grows. Furthermore, the head support section 196 is adjustable relative to the body-supporting portion of the inner layer 198. The relative positioning of the different layers 190, 196, 198 provides a large range of possible customization of the child-carrying pouch.

According to an embodiment, the head support section 196 and the inner layer 198 define a seat supporting section and separate head supporting section part of the child-carrying pouch 120. Accordingly, the seat supporting section (shown as the inner layer 198) is pre-molded to substantially match a shape of a child's buttocks area.

The child-carrying pouch 120 comprises a contour area 192 made of non-stretching material, and an inset area 194 inset to the contour area 192 that is made of stretchable material. The combination of non-stretching material for the contour area 192 and stretchable material for the inset area 194 fulfills the structural characteristics necessary to hold the child 30 in a secure manner, preventing openings between the harness portion 100 and the child-carrying pouch 120 to increase as the user 20 and the child 30 move, as fulfilling adjustment requirements for a growing child 30 as distinct shapes for the inner layer 198.

The child-supporting harness 210 further comprises e-textile comprising sensors 139 adapted to sense one or more of temperature, cardiac frequency and respiratory rate of the child 30 held in the child-carrying pouch 120. The sensors 139, according to an embodiment, are further adapted to sense one or more of the child's weight, the child's oxygenation level, the time spent in the carrier by the child 30, as information on the position of the child 30 in the child carrying pouch 120 (e.g. using pressure sensors) and movements of child 30 during the time spent in the child carrying pouch 120.

According to an embodiment, the e-textile, or more precisely the sensors 139, are located in the head support section 196 which is adapted to be in contact with the skin of the child 30. According to an embodiment, the sensors 139 are directly in contact with the skin of the child 30. According to an embodiment, the layer comprising the e-textile comprises a strip 138 extending from the main area of the layer and adapted to be inserted in the clothes of the child 30 via an opening (e.g. the neck opening or the sleeve opening) with the clothes participating in maintaining the strip 138 comprising sensors 139 in contact with the skin of the child 30.

One must note that even though the sensors 139 are illustrated localized in the strip 138, other and/or alternative locations where the sensors 139 may be used to collect information from the child 30. The illustrated location in the strip 138 (FIG. 23) reflects a particular embodiment wherein the sensor-bearing component is moveable and furthermore possible to attach to ensure contact to the child 30. Such an embodiment is exemplary and should not be construed as limiting the taught embodiments.

According to an embodiment, different sensors 139 are located at different locations of the child carrying pouch 120, the different sensors 139 providing similar information and complementary information that combined together provide useful data (e.g. pressure data combined to provide weight and position data). The collected data, regardless their nature, can therefore be used to participate in a m-Health process.

According to an embodiment, sensors 139 are electronic components embedded in the textile of the child carrying pouch 120 and are connected to user-owned electronic devices, devices specific to the monitoring process, or other components. According to an embodiment (not shown), touch buttons are constructed completely in textile forms by using conducting textile weaves, which are then connected to devices that are mounted on woven conducting fiber networks to form, for example, displays.

According to an embodiment, sensors 139 printed in the textile for both physiological and environmental monitoring are integrated into the textile of the child carrying pouch 120, for example cotton, polyester, nylon, Gore-Tex™, and neoprene. Therefore, accordingly, the sensors 139 are embedded in the yarns of at least some of the textiles composing the child carrying pouch 120.

According to an embodiment, the e-textile comprises components adapted to transform vibrations resulting from movements of the user 20 into power to recharge the power source powering the sensors 139.

According to an embodiment, the child-supporting harness 210 comprises, located according to one embodiment about one of the shoulder straps 110, a signalization device, e.g. a haptic component 140 which is, according to one embodiment, a vibration device 142. The haptic component 140 comprises components for communicating with one of the e-textile and a monitoring device 222 and to enter in an alarm mode when the sensors of the e-textile are monitoring conditions that meet an alarm criterion, or in other words are out of, above, or below, an acceptable range. For instance, the out-of-range data may comprise a respiratory rate out of acceptable range (too fast or too slow), a temperature out of acceptable range (too high or too low), and a cardiac frequency out of acceptable range (too fast or too slow). Further analysis may comprise combination of data out of acceptable range, or sudden changes in the monitored data, with the acceptable range limits being set accordingly.

According to another embodiment, the signalization device comprises, alone or in combination with the haptic component 140, a lighting device generating a light signal, and a speaker generating a sound signal. Accordingly, the signalization device generates a signal detectable by the user 20 to be informed of an entry in the alarm mode.

According to one embodiment, a processing component 226, for example a printed circuit board or PCB, is embedded with the sensors or the haptic component 140. The processing component 226 comprises a power source 224 and a memory 228 to store and process monitored data, and to trigger an alarm condition based on the processing of the monitored data. Accordingly, upon trigger of an alarm condition, the haptic component 140 would operate to transmit a signal recognizable by the user 20, for instance the vibration produced by the vibration device 142.

According to one embodiment, the sensors and the haptic component 140 are connected through wires to be interconnected when coupling the fixations 180, 182 or embedded in the fixations 180, 182 themselves. According to an embodiment, the sensors and the haptic component 140 are communicating wirelessly.

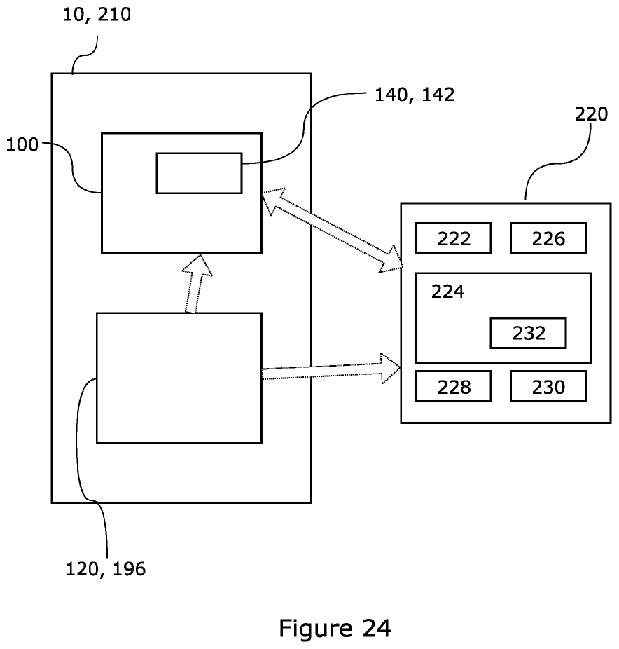

Referring additionally to FIG. 24, according to an embodiment the e-textile comprises communication components 230 enabling the e-textile to communicate monitored data to a monitoring device 222 (e.g. a smart phone) with a program 232 (e.g. an app) designed to receive the monitored data from the e-textile, store the data, and process the data. According to embodiments, the monitored data, or diagnostic data (processed monitored data) are available for the user 20 and health professionals to detect medical conditions of the child 30 and therefore be used to participate in a m-Health process. According to an embodiment, the haptic component 140 receives alarm condition signals from the monitoring device 222.

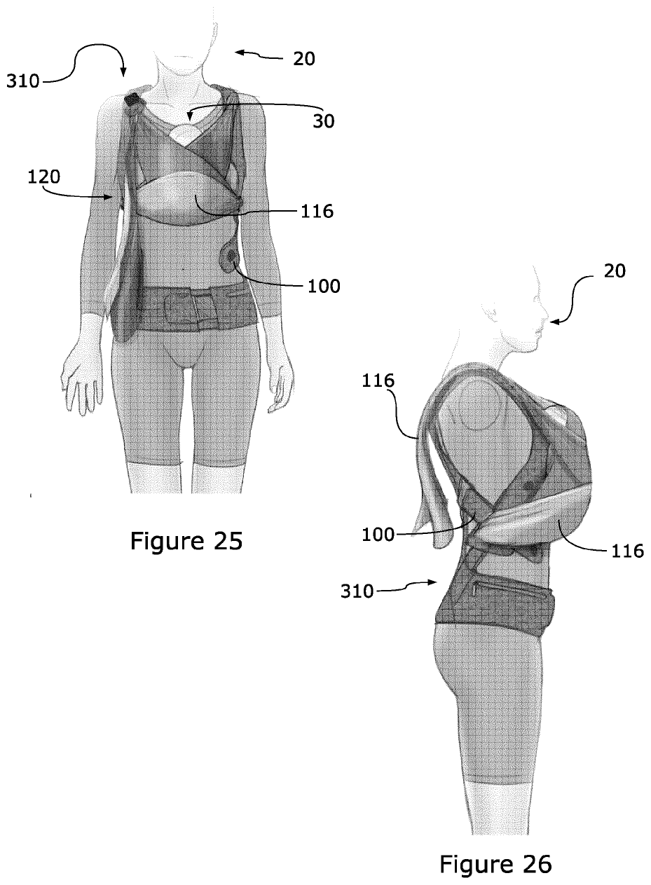

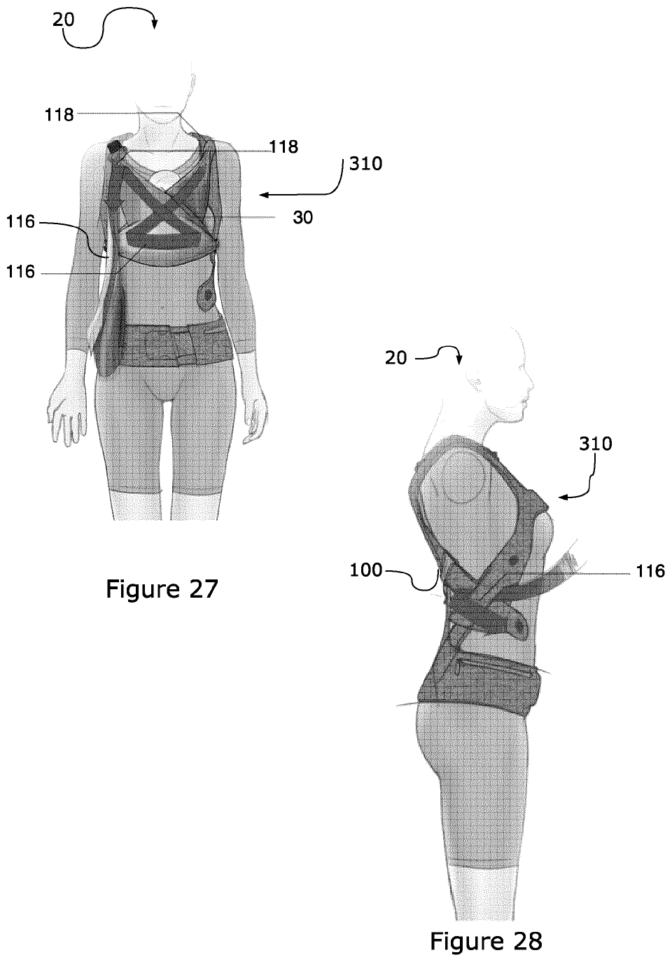

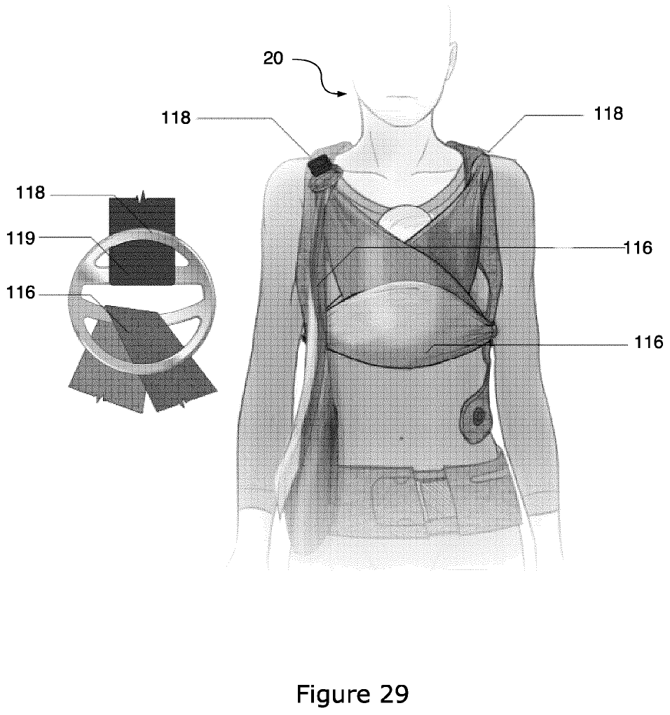

Referring now to FIGS. 25-29, another embodiment of a child-supporting harness 310 comprises a harness portion 100 and a shawl 116 shaped in a particular configuration to provides the functionalities of the child-carrying pouch 120. The shawl 116 is secured to fittings 118 (see FIG. 29) to be secured to the harness portion 100. In order to shape the child-carrying pouch 120, the user 20 attaches a first part of the shawl 116 to a first fitting 118 secured to the harness portion 100, and afterwards attach subsequent parts of the shawl 116 to other fittings 118 as illustrated on FIGS. 27 and 28 (wherein the arrow is illustrating a normal course of the shawl 116 when attached to the harness portion 110). The last section of the shawl 116 to be attached passes in the slot 176 on one side of the user 20, about the crossing to the pectoral portion 170 of the shoulder strap 110 with the torso strap 172. The last passage of the shawl 116 in the slot 176 blocks the final portion of the shawl 116 in place.

Referring particularly to FIG. 29, the fittings 118 used with a shawl 116 differ from the complementary fixations 182 used in relation with the previous embodiment while, according to an embodiment, being compatible with the latter. In embodiments, the fittings 118 further vary depending on the shawl 116 being attached to them at the beginning if its course or further during the attaching process. For instance, the illustrated fitting 118 on FIG. 29 comprises a strap 119 comprising a complementary fixation 182 (see FIG. 19) to be secured to a base fixation 182 (see FIG. 19).

According to embodiments, the shawl 116 comprises a series of layers and/or sections specifically designed for specific requirements. According to embodiments, some sections of the shawl 116 are reinforced while other section are designed with removable layers.

According to embodiments, the harness portion 100 is adapted to be used with distinct embodiments of child-carrying pouch 120, for example the one illustrated as part of the child-supporting harness 210 and the shawl 116 part of the child-supporting harness 310. The harness portion 100 is designed to adapt to the different weights and sized of the child 30, thus allowing a user 20 to select the best child-supporting compartment 120 as the child 30 grows older, the structure and fixations 180 of the support portion 100 being adapted for such a variety of conditions. It must be noted that components such as the power source 224, the processing component 226, the memory 228, the communication component 230 and the program 232 are illustrated part of the monitoring device 222 on FIG. 24, one must understand that, depending on embodiments, one or more of the above-identified components may be present in or in combination with the haptic component 140 and/or the e-textile, therefore providing a different level of autonomy to the component and allowing distinct communication channels as discussed herein before. Thus, locations of components as shown on FIG. 24 are a non-limiting example of combination and should not limit the scope the teaching of the present document.

It must be noted that, according to an embodiment, the textile selected to compose the child-supporting harness 10/210/310 are selected to be easy to clean, to be adapted for contact with the body of the user 20 and of the child 30 and therefore to be able to breath, to be in good part possible to dislodge from the fabric some parts less adapted to be cleaned (for example not adapted to be cleaned in a washing machine) with the other fabric able to be cleaned according to standard maintenances processes (such as in a washing machine). Some parts are able to be separated and to be secured together after a cleaning process to ease the cleaning process and/or the drying process.

While preferred embodiments have been described above and illustrated in the accompanying drawings, it will be evident to those skilled in the art that modifications may be made without departing from this disclosure. Such modifications are considered as possible variants comprised in the scope of the disclosure.

1. A child-supporting harness comprising:
a harness portion comprising:
a dorsal member having an upper section and a lower section;
a waist strap depending from the lower section of the dorsal member;
a pair of shoulder straps depending from the upper section of the dorsal member;
a pair of torso straps selectively attached to the dorsal member between the upper section and the lower section thereof; and
a child-carrying pouch releasably secured to the harness portion.

2. The child-supporting harness of claim 1, wherein the pair of torso straps is selectively attached to the dorsal member at a position which is adjustable between the lower section and the upper section of the dorsal member.

3. The child-supporting harness of claim 1, wherein each torso strap of the pair of torso straps crosses a corresponding shoulder strap of the pair of shoulder straps.

4. The child-supporting harness of claim 3, wherein one of the shoulder straps and the torso straps comprise a slot, with the other of the shoulder straps and the torso straps passes through the slot when crossing each other.

5. The child-supporting harness of claim 1, wherein each torso strap of the pair of torso straps comprises a free end which is unattached to other parts of the harness portion.

6. The child-supporting harness of claim 5, where the free end of each torso strap of the pair of torso straps comprises a base fixation used to releasable secure the child-carrying pouch.

7. The child-supporting harness of claim 1, wherein the child-carrying pouch comprises a seat support section and separate head support section.

8. The child-supporting harness of claim 7, wherein the seat support section is pre-molded to substantially match a shape of a child's buttocks area.

9. The child-supporting harness of claim 7, wherein the separate head support section is adjustable at least in height relative to the seat support section.

10. The child-supporting harness of claim 1, further comprising sensors for sensing a biometric parameter of a child in the child-carrying pouch.

11. The child-supporting harness of claim 10, further comprising a strip comprising the sensors for contacting the skin of the child when in the child-carrying pouch.

12. The child-supporting harness of claim 11, wherein the head supporting section comprises a main area and the strip, the strip extending from the main area and comprising the sensors, wherein the strip is for contacting the skin of the child when in the child-carrying pouch.

13. The child-supporting harness of claim 10, further comprising a signalization device located in the harness portion and in communication with the sensors, the signalization device generating a signal when at least one of the temperature, the cardiac frequency and the respiratory rate meets an alarm criterion.

14. The child-supporting harness of claim 13, wherein the signalization device comprises one of a vibration device generating a vibration signal, a lighting device generating a light signal, and a speaker generating a sound signal.

15. The child-supporting harness of claim 14, wherein the vibration device is located in at least one of the shoulder straps.

16. The child-supporting harness of claim 1, wherein the dorsal member comprises one of rigid material and semi-rigid material.

17. The child-supporting harness of claim 1, wherein the dorsal member comprises a first section and a second section, and a length, and wherein the first section is adjustable relative to the second section to adjust the length of the dorsal member.

18. The child-supporting harness of claim 1, wherein the harness portion comprises a front and a back which respectively correspond a front and a back of a user wherein the child-supporting harness further comprises additional base fixations located on the front and on the back of the harness portion, thereby allowing the child-carrying pouch to be secured both to the back and to the front of the harness portion.

19. The child supporting harness of claim 18, further comprising another child-carrying pouch releasably secured to the harness portion, thereby resulting in having one child-carrying pouch releasably secured to the front of the harness portion and the other child-carrying pouch releasably secured to the back of the harness portion.

20. The child-supporting harness of claim 1, wherein the child-carrying pouch comprises a seat supporting section and a sitting-width about the seat-supporting section, wherein the child-carrying pouch comprises an adjusting means to adjust the sitting-width of the child-carrying pouch.

21. (canceled)
